(19) United States
(12) Patent Application Publication
(10) Pub. No.: US 2025/0223647 A1
(43) Pub. Date: Jul. 10, 2025

(54) RNA-BASED METHOD FOR STROKE ASSESSMENT AND TREATMENT

(71) Applicant: Morehouse School of Medicine, Atlanta, GA (US)

(72) Inventor: Robert MELLER, Covington, GA (US)

(73) Assignee: Morehouse School of Medicine, Atlanta, GA (US)

(21) Appl. No.: 19/015,231

(22) Filed: Jan. 9, 2025

Related U.S. Application Data
(60) Provisional application No. 63/619,066, filed on Jan. 9, 2024.

(51) Int. Cl. C12Q 1/6883

(52) U.S. Cl. CPC C12Q 1/6883; C12Q 2600/106; C12Q 2600/158

(57) ABSTRACT

A method for screening stroke suspects, comprising the steps of extracting RNA from a blood sample of a stroke suspect, generating a transcriptome profile of the stroke suspect based on the RNA extracted from the blood sample, analyzing the transcriptome profile of the stroke suspect to generate a report indicating whether the stroke suspect suffered a stroke and, in the case that the stroke suspect suffered a stroke, whether the stroke is an ischemic stroke or a hemorrhagic stroke.

### RELATED APPLICATION

This application claims priority of U.S. Provisional Application Ser. No. 63/619,066, filed on Jan. 9, 2024. The contents of the aforementioned application are incorporated herein by reference.

This application was made with government support under grant nos. NS112422 and MD007602 awarded by the National Institutes of Health. The government has certain rights in the application.

### FIELD

The present disclosure relates generally to medical treatment and, in particular, to an RNA-based method for stroke assessment and treatment.

### BACKGROUND

A stroke occurs when the blood supply to brain tissue is blocked by a blood clot (ischemic stroke), or when a blood vessel in the brain ruptures (hemorrhagic stroke), causing brain cells to die and leading to functional impairments. Stroke is a leading cause of death and disability both globally and in the U.S., where approximately 800,000 people experience a stroke each year.

Tissue plasminogen activator (tPA) is a serine protease that can dissolve blood clots that block blood flow to the brain. Treatment with tPA, however, was associated with a greater risk of bleeding in the brain and, therefore, should not be used in patients with hemorrhagic stroke.

Typically, patients presenting stroke symptoms are imaged using CT imaging, the results are interpreted and reviewed by a radiologist and neurologist, prior to tPA being administered. This procedure is performed to exclude any patients with hemorrhagic stroke from receiving tPA. This procedure causes significant delay, especially in non-stroke specialist medical facilities. Many patients do not receive tPA following stroke because hemorrhagic stroke cannot be excluded within the optimal time window for tPA treatment.

Therefore, there is a need for the development of a rapid test that is capable of differentiating ischemic stroke from hemorrhagic stroke. Such a test would enable tPA therapy decisions to be made in a timely manner.

### SUMMARY

One aspect of the present application relates to a method for assessing stroke suspects. The method comprises the steps of extracting RNA from a blood sample of a stroke suspect, generating a transcriptome profile of the stroke suspect based on the RNA extracted from the blood sample, and analyzing the transcriptome profile of the stroke suspect to generate a report indicating whether the stroke suspect suffered a stroke and, in the case that the stroke suspect suffered a stroke, whether the stroke is an ischemic stroke or a hemorrhagic stroke.

In some embodiments, the blood sample is collected at admission of a medical facility. In some embodiments, the RNA is extracted from the whole blood.

In some embodiments, the analyzing step comprises comparing the transcriptome profile of the stroke suspect to established transcriptome profiles of stroke patients and determining the stroke subtype.

In some embodiments, the report indicates that the stroke suspect suffered a stroke and includes a recommendation for appropriate treatment or prevention regime for the stroke.

In some embodiments, the method further comprises the step of treating the stroke suspect with a stroke treatment, when the report indicates that the stroke suspect suffered a stroke.

In some embodiments, the method further comprises the step of treating the stroke suspect with tPA or performing mechanical clot removal, when the report indicates that the stroke suspect suffered an ischemic stroke.

In some embodiments, the method indicates that the stroke suspect suffered a hemorrhagic stroke and tPA is contraindicated.

Another aspect of the present application relates to a method for treating stroke patients with tPA. The method comprises the steps of extracting RNA from a blood sample of a stroke suspect, generating a transcriptome profile of the stroke suspect based on the RNA extracted from the blood sample, comparing the transcriptome profile of the stroke suspect to an established transcriptome profile of ischemic stroke patients; determining whether the stroke suspect suffered an ischemic stroke based on a result of the comparing step; and treating the stroke suspect with tPA, if the stroke suspect suffered an ischemic stroke.

In some embodiments, the stroke suspect is deemed to have suffered an ischemic stroke, when the transcriptome profile of the stroke suspect matches the established transcriptome profile of ischemic stroke patients.

In some embodiments, the method further comprises the step of comparing the transcriptome profile of the stroke suspect to an established transcriptome profile of hemorrhagic stroke patients and the stroke suspect is deemed to have suffered an ischemic stroke, when (1) the transcriptome profile of the stroke suspect matches the established transcriptome profile of ischemic stroke patients and (2) the transcriptome profile of the stroke suspect does not matches the established transcriptome profile of hemorrhagic stroke patients.

### BRIEF DESCRIPTION OF THE DRAWINGS

FIG. 1 shows an overview of adjunct blood transcriptome test to assess stroke.

FIG. 2 shows correlation analysis of RNA-Seq libraries independently created from the same RNA samples.

FIG. 3 shows assessment of read depth with identification of transcripts. Blood cells appear to plateau at 50% of transcripts in the model once over 5 million aligned reads.

FIG. 4 shows differential RNA expression of ischemic vs hemorrhagic stroke in blood obtained at admission from GMH. Differential RNA expression is determined by ANOVA, using Partek Genomics suit. Panel A. Data shown are RPKM normalized counts, using hemorrhage (no tpa) stroke or stroke mimic category as contrast factors. Data are FDR p,0.05, for 1.5 fold changes. Panel B. Differentially expressed genes were subjected to PCA to show clustering of different samples by type.

FIG. 5 shows differential RNA isoform expression of ischemic vs hemorrhagic stroke in blood obtained at admission from GMH. Differential RNA transcript expression is determined by ANOVA, using Partek Genomics suit. Panel A. Data shown are RPKM normalized counts, using hemorrhage (no tPA) stroke or stroke mimic category as contrast factors. Data are unadj p<0.001, for 1.2 fold changes. Panel B. Differentially expressed transcripts were subjected to PCA to show clustering of different samples by type. Of note the same samples cluster with no-tPA as in FIG. 6.

FIG. 6 shows differential RNA expression of stroke vs stroke mimics in blood obtained at admission from GMH. Differential RNA expression is determined by ANOVA, using Partek Genomics suit. Data shown are RPKM normalized counts, using stroke or stroke mimic category as contrast factors (Panel A). Data are p<0.001, for 1.2-fold changes. Differentially expressed genes were subjected to PCA to show clustering of different samples by sample type (Panel B).

FIG. 7 shows differential RNA expression in different stroke subtypes (TOAST) in blood obtained at admission from GMH. Differential RNA expression is determined by ANOVA, using Partek Genomics suit. Data shown are RPKM normalized counts, using stroke or stroke mimic category as contrast factors. Data are p<0.001, for 1.2-fold changes. Panel A. Differentially expressed genes were subjected to PCA to show clustering of different samples by sample type. Panel B. Same data as left, but with expression values of TIA samples overlaid.

FIG. 8A-8D shows sex-specific gene expression profiles may be more accurate than a mixed sex model. Data from a previous study (Meller et al., Ann Clin Trans Neurol. 3:70-81, 2016) was refined, and only age and sex matched MCA stroke and controls used. Panel A. Hierarchical clusters of differentially expressed exon values in male only participants. Panel B. Hierarchical cluster of differentially expressed exon values in female only group. Panel C. Venn diagram showing overlap of exons in models identifying mixed sex, or sex specific changes following MCA. Note the lack of common genes in sex-specific populations. Panel D. Power analysis modeling of sex-specific data (female) to determine sample size (x axis) and power (y axis) to detect 2-fold changes with FDR 0.05 (modeled in SPAA).

FIG. 9A-9B shows differential gene expression in tPA treated and untreated stroke patients with respect to change in stroke severity. Panel A DEG expression values were subjected to hierarchical clustering (above) and principal component analysis in tPA treated and untreated patients. Panel B When the DEG lists were combined PCA reduction shows a separation of tPA treated samples, but not untreated with respect to change in stroke symptom severity.

While the present disclosure will now be described in detail, and it is done so in connection with the illustrative embodiments, it is not limited by the particular embodiments illustrated in the figures and the appended claims.

### DETAILED DESCRIPTION

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

### I. Definitions

As used herein, the following terms have the meanings ascribed to them unless specified otherwise:

The term “library” as used herein, refers to a collection of polynucleotides derived from nucleic acid sequences of a particular tissue, in particular RNA or cDNA. The polynucleotides of a library may be, but are not necessarily, cloned into a vector or set in a microarray.

The terms “nucleic acid” “polynucleotide” and “oligonucleotide” may be used interchangeably herein and refer to a deoxyribonucleotide or ribonucleotide polymer in either single- or double-stranded form. A “subsequence” or “segment” refers to a sequence of nucleotides that comprise a part of a longer sequence of nucleotides.

A “gene,” for the purposes of the present disclosure, includes a DNA region encoding a gene product. The region can also include DNA regions that regulate the production of the gene product, whether or not such regulatory sequences are adjacent to coding and/or transcribed sequences. This term in science also encompasses RNAs which are expressed by a cell, but that are not translated into a protein, such as a non-coding RNA, micro RNA, piRNA, etc. Accordingly, a gene can include, without limitation, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA, or a novel RNA whose function is as yet to be determined) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

The term “transcriptome” or “whole transcriptome” refers to the set of all RNA molecules, including both coding and non-coding RNAs found in one cell or found in a population of cells. It is herein used to refer to all RNAs unless otherwise stated (e.g., the transcriptome is all RNA species, and their parts such as different isoforms (transcripts) and exons (small parts)). The transcriptome differs from the exome in that the transcriptome consists of only those RNA molecules contained in a specified cell population, and normally concerns the amount or concentration of each RNA molecule in addition to their molecular identities. In contrast to the genome, the transcriptome can vary with external environmental conditions. Since the transcriptome comprises all RNA transcripts in the cell, the transcriptome reflects the active expression of different genes at any given time.

The term “non-coding RNA” (ncRNA) refers to an RNA molecule that is not translated into a protein. The number of non-coding RNAs within the human genome is unknown; however, recent transcriptomic and bioinformatics studies suggest that there are thousands of them. Many of the newly identified ncRNAs have not been validated for their function. It is also likely that many ncRNAs are non-functional (sometimes referred to as junk RNA), and are the product of spurious transcription. Abundant and functionally important types of non-coding RNAs include transfer RNAs (tRNAs) and ribosomal RNAs (rRNAs), as well as small RNAs such as microRNAs, siRNAs, piRNAs, snoRNAs, snRNAs, exRNAs, scaRNAs and the long ncRNAs such as Xist and HOTAIR. The ncRNA may have some associated activity that may be deleterious. Most often the major concern is whether it will be translated into short random peptides.

### II. Method for Assessing and Treating Stroke Patients

One aspect of the present application relates to a method to assess whether a stroke suspect has experienced a stroke and if so, to identify the cause of the stroke. In some embodiments, the method may further comprise the step of treating the stroke suspect with an appropriate treatment if the suspect suffered a stroke.

In some embodiments, the method comprises the steps of (1) extracting RNA from a blood sample of a stroke suspect, (2) generating a transcriptome profile based on the RNA extracted from the blood sample, and (3) analyzing the transcriptome profile to generate an outcome indicating whether the stroke suspect experienced a stroke and, in the case that the stroke suspect experienced a stroke, the cause of the stroke. The method is utilized to differentiate ischemic stroke from hemorrhagic stroke and may serve as a screen tool for candidates of tPA treatment.

In some embodiments, the method comprises the steps of extracting RNA from a blood sample of a stroke suspect, generating a transcriptome profile based on the RNA extracted from the blood sample, analyzing the transcriptome profile to generate an outcome indicating whether the stroke suspect is a candidate of tPA treatment. In some embodiments, the method further comprises the step of treating the stroke suspect with tPA if the outcome of the analyzing step indicates that the suspect is a candidate of tPA treatment.

### Stroke Suspect

A stroke suspect is a subject who is suspected of experienced a stroke. In some embodiments, the stroke suspect is a human.

In some embodiments, the stroke suspect exhibits one or more symptoms of stroke. Examples of stroke symptoms include, but are not limited to, sudden numbness or weakness in the face, arm, or leg—especially on one side of the body; sudden confusion, trouble speaking, or understanding speech; sudden problems seeing in one eye or both eyes; and sudden dizziness, loss of balance or coordination, or trouble walking. While exhibiting stroke symptoms, the stroke suspect may not suffered a stroke (such stroke suspects are also referred to as “stroke mimics” in this application

In some embodiments, the stroke suspect is identified as at risk of a stroke based on a prehospital and/or hospital stroke screening method. Examples of such screening method include, but are not limited to, Cincinnati Pre-hospital Stroke Scale (CPSS), Face Arm Speech Test (FAST), Recognition of Stroke in the Emergency Room (ROSIER) and Los Angeles Pre-hospital Stroke Screen (LAPSS).

In some embodiments, the stroke suspect is exhibiting symptoms of, or is suspected of having, an ischemic stroke. In some embodiments, the subject has experienced and/or is at risk of having an intracerebral hemorrhage or hemorrhagic stroke.

In some embodiments, the stroke suspect is asymptomatic but has a risk or predisposition to experiencing stroke, e.g., based on genetics, a related disease condition, environment or lifestyle. For example, in some embodiments, the patient suffers from a chronic inflammatory condition, e.g., has an autoimmune disease (e.g., rheumatoid arthritis, Crohn's disease inflammatory bowel disease), atherosclerosis, hypertension, or diabetes. In some embodiments, the patient has high LDL-cholesterol levels or suffers from a cardiovascular disease (e.g., atherosclerosis, coronary artery disease). In some embodiments, the patient has an endocrine system disorder, a neurodegenerative disorder, a connective tissue disorder, or a skeletal and muscular disorder.

### Sample Collection and RNA Extraction

The blood sample of the stroke suspect should be collected as soon as possible after the suspected stroke, and as soon as the patient is under medical care including EMT, primary care practice or following admission to an emergency room. In some embodiments, the blood sample is collected upon admission at a medical facility. In some embodiments, the blood is collected with the PAXgene blood collection tubes. A whole blood sample contains six types of cells: red blood cell, neutrophil, eosinophil, basophil, lymphocyte, and monocyte, as well as platelets.

RNA is extracted from the blood sample using methods well known in the art. Briefly, the blood sample is treated with reagents that lyse blood cells and inactivate cellular RNases. Cellular RNA is then isolated and subjected to further analysis. Examples of RNA extraction kits include, but are not limited to, Tempus blood RNA isolation system (ThermoFisher Scientific), RiboPure-Blood kit (ThermoFisher Scientific), LeukoLOCK (ThermoFisher Scientific), and QiaCube (Qiagen). In some embodiments, the RNA is extracted with Qiatube from a whole blood sample. In some embodiments, the RNA is from a whole blood sample. In some embodiments, one or more specific cell components of the blood, such as red blood cells, neutrophils, eosinophils, basophils, lymphocytes, and/or monocytes, may be isolated or purified, prior to RNA extraction, or determined by single cell sequencing methodologies.

### Generation of Transcriptome Profile

RNA extracted from the blood sample is subjected to RNA-sequencing (also referred to as RNA-Seq) to generate a transcriptome profile of the stroke suspect. RNA-Seq uses high-throughput sequencing to illuminate the existence and relative quantities of RNA molecules at a given moment in a biological sample. In addition to mRNA transcripts, RNA-Seq can also look at different populations of RNA to include the whole RNS transcriptome (such as miRNA or tRNA).

RNA-Seq works in concert with a range of high-throughput DNA sequencing technologies. However, prior to sequencing of the extracted RNA transcripts, several key processing steps are performed. Methods differ in the use of transcript enrichment, fragmentation, amplification, single or paired-end sequencing, and whether to preserve strand information. One of ordinary skill will understand that the particular type or form of RNA-Seq is not limiting on the application discussed herein.

In the case of blood, the RNA extract may contain a large amount of ribosomal RNA (rRNA) and non-coding RNA (ncRNA). The sensitivity of any given RNA-Seq analysis can be enhanced by enriching RNA classes of interest, while depleting known abundant RNAs. If so desired, the mRNA molecules can be removed by using oligonucleotides probes that bind their poly-A tails or enriched by using primers with polyT sequences. Alternatively, abundant but uninformative ribosomal RNAs (rRNAs) can be removed by ribo-depletion by hybridisation to probes designed to target specific rRNA sequences (e.g. mammal rRNA, plant rRNA). However, ribo-depletion may also introduce some bias via non-specific depletion of off-target transcripts. Gel electrophoresis and extraction can be used to purify small RNAs, such as micro RNAs, by their size.

In a preferred embodiment, the RNA extract is subjected to RNA-seq without removal of rRNA or ncRNA, and without enrichment of mRNA. RNA-seq of such RNA extraction permits the identification of both coding and non-coding RNAs (whole transcriptome). Whole transcriptome analysis detects changes in exon expression and alternative transcript splicing events that occur rapidly following stroke, thus allowing more accurate biomarker panel profiling/RNA signature determination for stroke and stroke subtype differentiation.

In some embodiments, rRNA is removed from the RNA extract prior to RNA-Seq. In some embodiments, rRNA is not removed from the RNA extract prior to RNA-Seq. In some embodiments, rRNA is not removed from the RNA extract prior to RNA-Seq but rRNA sequences are bioinformatically removed following RNA-seq.

In some embodiments, ncRNA is removed from the RNA extract prior to RNA-seq. In some embodiments, ncRNA is not removed from the RNA extract prior to RNA-seq. In some embodiments, ncRNA is not removed from the RNA extract prior to RNA-Seq but ncRNA sequences are bioinformatically removed following RNA-seq.

In some embodiments, the RNA extract is enriched for mRNA prior to RNA-Seq. In some embodiments, the RNA extract is not enriched for mRNA prior to RNA-Seq.

In some embodiments, the RNA extract is subjected to RNA-seq without removal of rRNA or ncRNA, and without enrichment of mRNA. RNA-seq of such RNA extraction permits the identification of both coding and non-coding RNAs (whole transcriptome). Whole transcriptome analysis detects changes in exon expression and alternative transcript splicing events that occur rapidly following stroke, thus allowing more accurate biomarker panel profiling/RNA signature determination for stroke and stroke subtype differentiation. In some embodiments, ncRNA and/or rRNA sequences are bioinformatically removed following RNA-seq

In some embodiments, the extracted RNA is fragmented prior to RNA-Seq. Fragmentation may be achieved by chemical hydrolysis, nebulisation, sonication, or reverse transcription with chain-terminating nucleotides. Alternatively, fragmentation and cDNA tagging may be done simultaneously by using transposase enzymes. One of ordinary skill will understand that the particular method of preparing a transcriptome for sequencing is not limiting on the application discussed herein.

The extracted RNA can be sequenced in just one direction (single-end) or both directions (paired-end). A single-end sequence is usually quicker to produce, cheaper than paired-end sequencing and sufficient for quantification of gene expression levels. Paired-end sequencing produces more robust alignments/assemblies, which is beneficial for gene annotation and transcript isoform discovery. Strand-specific RNA-Seq, methods preserve the strand information of a sequenced transcript. Without strand information, reads can be aligned to a gene locus but do not inform in which direction the gene is transcribed. Stranded-RNA-Seq is useful for deciphering transcription for genes that overlap in different directions and to make more robust gene predictions in non-model organisms. One of ordinary skill will understand that the particular strands used in sequencing are not limiting on the application described herein.

The RNA-Seq may be performed using methods well known in the art. Examples of such methods include quantitative polymerase chain reaction (qPCR), high throughput multiplex nucleic acid sequencing and nanopore sequencing. In some embodiments, the RNA-Seq is perform with the Ion Torrent Platfrom (ThermoFisher). In some embodiments, the RNA extract is not primed with a polyT primer, thus reducing bias for polyA and 3′ transcripts. In some embodiments, the RNA-Seq libraries are stranded libraries.

### Transcriptome Assembly

The raw data generated by sequencing is then processed to generate a transcriptome profile for the seizure suspect. Transcriptomics methods are highly parallel and require significant computation to produce meaningful data for both microarray and RNA-Seq experiments. RNA-Seq analysis generates a large volume of raw sequence reads which have to be processed to yield useful information. Data analysis usually requires a combination of bioinformatics software tools that vary according to the experimental design and goals. The process can be broken down into four stages: quality control, alignment, quantification, and differential expression. Most popular RNA-Seq programs are run from a command-line interface, either in a Unix environment or within the R/Bioconductor statistical environment.

Sequence reads are not perfect, so the accuracy of each base in the sequence needs to be estimated for downstream analyses. Raw data is examined to ensure: quality scores for base calls are high, the GC content matches the expected distribution, short sequence motifs (k-mers) are not over-represented, and the read duplication rate is acceptably low. Several software options exist for sequence quality analysis, including FastQC and FaQCs. Abnormalities may be removed (trimming) or tagged for special treatment during later processes.

In order to link sequence read abundance to the expression of a particular RNA, transcript sequences are aligned to a reference genome. The key challenges for alignment software include sufficient speed to permit billions of short sequences to be aligned in a meaningful timeframe, flexibility to recognize and deal with intron splicing of eukaryotic mRNA, and correct assignment of reads that map to multiple locations. Software advances have greatly addressed these issues, and increases in sequencing read length reduce the chance of ambiguous read alignments. One of ordinary skill will understand the choice of high-throughput sequence aligners that are available and may be selected for analyses.

Alignment of primary transcript mRNA sequences derived from eukaryotes to a reference genome requires specialized handling of intron sequences, which are absent from mature mRNA. Short read aligners perform an additional round of alignments specifically designed to identify splice junctions, informed by canonical splice site sequences and known intron splice site information. Identification of intron splice junctions prevents reads from being misaligned across splice junctions or erroneously discarded, allowing more reads to be aligned to the reference genome and improving the accuracy of gene expression estimates. Since gene regulation may occur at the mRNA isoform level, splice-aware alignments also permit detection of isoform abundance changes that would otherwise be lost in a bulked analysis.

In some embodiments, RNA sequencing data (reads) are aligned to the human genome (Grch38) using a custom script combining STAR and Bowtie2 alignment software. In some embodiments the data are aligned or mapped with alternative software (e.g. minimap2, hisat, bwa, kraken, salmon etc). in some embodiments the data are aligned to complete end to end versions of the human genome (such as Chm-13 from the Telomere to telomere project), or ancestry specific reference genomes, or a human pan-genome model. Gene aligned reads (Counts data) are analyzed using Partek Genomics Studio. All data are normalized to reads per kilobase per million aligned reads, and all data are further normalized by dividing by the trimmed mean of the rkpm values. Gene expression and transcript usage is determined using linear models following correction for batch (in linear model). In some embodiments alternative normalization methods are utilized including counts, counts per million or transcript per million aligned reads approaches.

In some embodiments, custom annotation guides are created to quantify novel RNAs that align to previously unannotated regions of human genome. Briefly, a custom annotation guide is created using software to first identify the genomic origin of a detected RNA in the sequencing data. This creates a datafile (gtf or .gff) of genomic regions, which are compiled for all samples, and then compared and merged with the published available reference annotation guide using various software. This new annotation guide will contain all genomic regions that express RNA, which are then used by current RNA counting tools to derive the RNA expression.

### Determination of Stroke Cause Based on the Transcriptome Profile of the Stroke Suspect

The transcriptome profile of the stroke suspect is then compared to a database of stroke patient transcriptome profiles. The database further contains the transcriptome profiles of non-stroke individual transcriptome profiles, stroke mimics (individuals showing stroke symptoms but did not experience a stroke) transcriptome profiles. The stroke patient transcriptome profiles include, but are not limited to, general stroke transcriptome profiles that contain robust differentially expressed genes (DEGs) characteristic in all stroke patients, stroke subtype specific transcriptome profiles, such as hemorrhagic stroke specific transcriptome profiles and ischemic stroke specific transcriptome profiles. The ischemic stroke specific transcriptome profiles may further include cardio-embolic (MCA-C) stroke specific transcriptome profiles, large vessel thrombolic stroke (MCA-A) specific transcriptome profiles, atherosclerotic stroke specific transcriptome profiles, sub-cortical stroke specific transcriptome profiles and transient ischemic attack specific transcriptome profiles. In some embodiments, the transcriptome profiles are further divided into sub-transcriptome profiles characterized by sex, race, age, etc. to provide more accurate assessment of the transcriptome profile of the stroke suspect.

The raw sequencing data from these patients are analyzed using mathematical clustering to identify patterns of gene expression and transcriptomic signature that are unique for each stroke subtype, patient type and/or treatment type.

As shown in the Examples of this application, individuals who suffered a stroke have unique RNA expression patterns (also referred to as RNA signatures) that are different from individuals who have not suffered a stroke. In addition, individuals who suffered different types of stroke showed different RNA expression patterns, thus allowing differentiation of stroke subtype based on the RNA expression patterns. In some embodiments, the database further contains the biographical information, such age, sex, race, and marital status, as well as personal and family medical history of each individual. Such parameters may also be included in the algorithm for determining stroke subtype. In some embodiments, the database further contains blood transcriptome patterns of stroke patients who received treatment (such as tPA treatment). Such information may be used to evaluate treatment efficacy and safety of the treatment and may also serve as a predictor of therapeutic responses.

In some embodiments, the database of stroke patient transcriptome profiles contains a database on predictive transcriptomic signatures that may serve as predictors of treatment outcome. The database of stroke patient transcriptome profiles may be updated from time to time with new data using artificial-intelligence and machine learning tools to improve accuracy of the diagnosis.

Comparison of the transcriptome profile of the stroke suspect to the stroke RNA profiles allow for the determination of (1) whether the stroke suspect suffered a stroke, and (2) if the stroke suspect suffered a stroke, the subtype of the stroke. In a particular embodiment, the comparison of the transcriptome profile of the stroke suspect to the stroke RNA profiles allows for the differentiation of ischemic stroke from hemorrhagic stroke.

In some embodiments, the stroke suspect is deemed to have suffered a stroke, if the transcriptome profile of the stroke suspect matches a reference transcriptome profile of stroke patients. A match is made when the profile is compared to a prediction model derived from a training dataset of RNA profiles for which the clinical diagnosis (stroke phenotype) is known. Once a model is identified with a high accuracy and sensitivity/specificity, it is then used to call a prediction on the test sample. The prediction may be a classifier value such as stroke/non-stroke, or a numerical value such as time following stroke. A multi factorial prediction model may be able to determine a factor that is greater than binary, for example ischemic stroke vs hemorrhagic stroke phenotype, or a recommended drug to treat the patient with. Prediction models are trained using common software packages and may involve linear/non-linear approaches or Machine Learning Artificial Intelligence algorithms. In some embodiment, the algorithm further provides a proposed treatment regimen based on the suspect's transcriptome profile, biographic information, person medical history and family medical history.

In some embodiments, the algorithm is capable of (1) discriminating between ischemic and hemorrhagic stroke, (2) identifying the timing of stroke occurrence, and/or (3) predicting hemorrhagic transformation risk.

In some embodiment, the algorithm further provides a proposed treatment regimen based on the suspect's transcriptome profile, biographic information, person medical history and family medical history. For example, our experimental data shows that tPA treatment appear to be less effective in female African Americans who suffered ischemic stroke.

In some embodiments, determination of stroke occurrence is based on the expression level of a panel of stroke-associated biomarkers. As used herein, the term “stroke-associated biomarkers” refers to genes that are either over-expressed or under-expressed in stroke patients comparing to the expression level of the same markers in otherwise healthy individuals (e.g., in individuals who have not experienced and/or are not at risk of experiencing stroke). The stroke patient database maintains and updates a database of stroke-associated biomarkers, as well as a database of stroke-subtype associated biomarkers. As used herein, the term “stroke subtype-associated biomarkers' refers to genes that are either over-expressed or under-expressed in patients suffered from the corresponding subtype of stroke comparing to the expression level of the same markers in otherwise healthy individuals (e.g., in individuals who have not experienced and/or are not at risk of experiencing stroke). Stroke-subtype associated biomarkers include, but are not limited to, ischemic stroke-associated biomarkers and hemorrhagic stroke-associated biomarkers.

In some embodiments, the overexpression or under-expression of stroke-associated biomarker/stroke-subtype associated biomarkers is determined with reference to the expression level of the same ischemic stroke-associated biomarker in an otherwise healthy individual. For example, a healthy or normal control individual has not experienced and/or is not at risk of experiencing ischemic stroke. The healthy or normal control individual generally has not experienced a vascular event (e.g., ischemic stroke, myocardial infarction, peripheral vascular disease, or venous thromboembolism). The healthy or normal control individual generally does not have one or more vascular risk factors (e.g., hypertension, diabetes mellitus, hyperlipidemia, or tobacco smoking). As appropriate, the expression levels of the target ischemic stroke-associated biomarker in the healthy or normal control individual can be normalized (i.e., divided by) the expression levels of a plurality of stably expressed RNA reference expression blood profile biomarkers.

In some embodiments, the term “over-expression” refers to an expression level that is 50%, 100%, 200%, 500%, or 1000% greater than the reference expression level. In some embodiments, the term “under-expression” refers to an expression level that is less than 50%, 20%, 10%, 5%, or 1% of the reference expression level. In some embodiments, the reference expression level of a gene is the expression level of the gene in an individual who has not experienced and/or are not at risk of experiencing stroke.

In some embodiments, the method of the present application further comprises the step of providing a recommendation for treatment and/or prevention regimes to a patient diagnosed as having a stroke or at risk of the occurrence of a stroke. Such recommendation may include medications (e.g., tPA) and life-style adjustments (e.g., diet, exercise, stress) to minimize risk factors such as high blood pressure and cholesterol levels, and control diabetes.

In some embodiments, the method of the present application is used as an adjuvant to CT imaging or even replace imaging for determination of whether to give tPA treatment to the stroke suspect.

In some embodiments, the method of the present application is used to predict response to therapy (e.g., tPA treatment or mechanical removal of clot) in the stroke suspect.

### III. Data Analysis System and Program Product

As will be appreciated by one of skill in the art, method of the present application may be embodied as a data analysis system or program products. Accordingly, the method of the present application may take the form of data analysis systems or data analysis software, etc. Software written according to the present application is to be stored in some form of computer readable medium, such as memory, hard-drive, DVD ROM or CD ROM, or transmitted over a network, and executed by a processor. One aspect of the present application provides a computer system for analyzing data from the transcriptome of a blood sample of a stroke suspect, and determining time of stroke occurrence, subtype of stroke, physiological status of the stroke suspect, potential treatment regimen and/or therapeutic efficacy. The computer system comprises a processor, and memory coupled to said processor which encodes one or more programs. The programs encoded in memory cause the processor to perform the steps of the above methods wherein the expression profiles and information about physiological, pharmacological and disease states of the stroke suspect are received by the computer system as input. The program encoded in memory also causes the computer to access the stroke patient database in order to perform analysis as described in the analysis step described above to generate an outcome.

Another aspect of the present application provides a server that harbors the database and the program for carrying out the methods of the present application.

The present application is further illustrated by the following examples that should not be construed as limiting. The contents of all references, patents, and published patent applications cited throughout this application, as well as the Figures and Tables, are incorporated herein by reference.

### EXAMPLES

### Example 1: RNA-Seq can Diagnose Patients Who have Suffered a Stroke

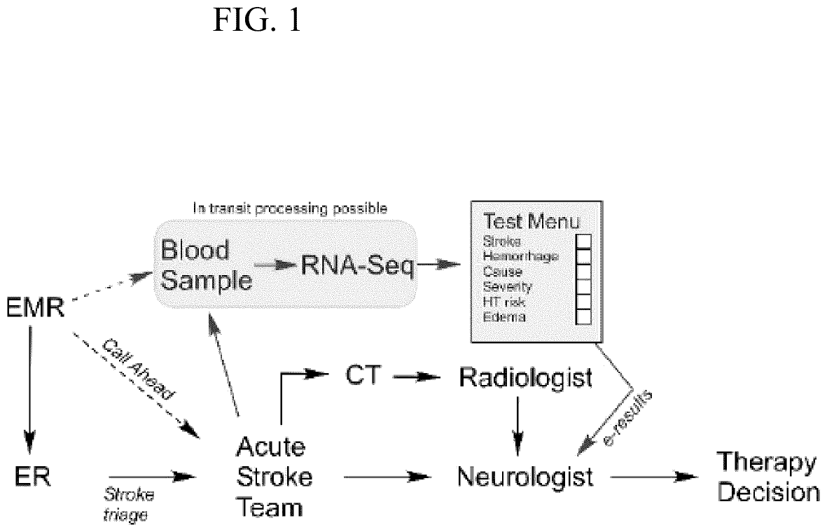

This study utilizes whole transcriptome profiling to identify RNA signatures in whole blood following stroke (FIG. 1). A pipeline to extract RNA from whole blood samples and perform whole transcriptome analysis has been established. This study assesses the whole transcriptome and not just protein encoding mRNAs. RNA is not primed with a polyT primer, reducing bias for PolyA and 3′ transcripts. In addition, the libraries are stranded. The use of custom annotation guides helps to quantify novel RNAs that align to previously unannotated regions of the human genome.

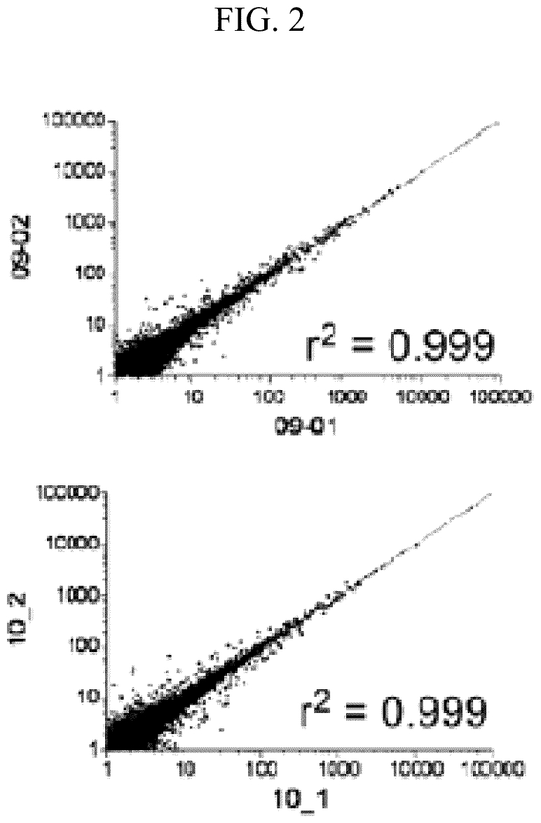

The study compared transcriptomes from two sets of libraries assembled using the same RNA (FIG. 2). Briefly, both principal component analysis and individual correlation analysis were performed on two independently built RNA-Seq libraries: r2 values of 0.999, showing RNA-Seq is reliable. Protocols for the assembly and sequencing of libraries were established, and RNA spike in controls (ERCC) enabled an assessment of library building between sample batches.

The study further investigated the ability of exon expression levels (reads/kilobase per million aligned reads (RKPM)) to identify patients with middle cerebral artery (MCA) ischemic stroke from controls. Following statistical analysis, data were subjected to hierarchical cluster analysis and principal component analysis (PCA). The result suggests that RNA-Seq can diagnose patients who have suffered a stroke.

This approach focuses on the application of mathematical clustering to identify patterns of gene expression predictive of diagnosis rather than an investigation of gene expression per se, nor gene biological consequence(s). This approach is sensitive, and can discriminate between strokes of different etiology, such as cardio-embolic (MCA-C) and large vessel thrombotic stroke (MCA-A).

### Example 2: Blood RNA-Seq can Discriminate Between Ischemic and Hemorrhagic Stroke

The study reanalyzed all stroke patient data to determine stroke subtype. The study observes a clear separation of RNA profiles associated with hemorrhagic stroke versus ischemic stroke (cardioembolic, atherosclerotic, sub-cortical stroke, and transient ischemic attack: TIA), or were deemed to have not suffered stroke (stroke mimic).

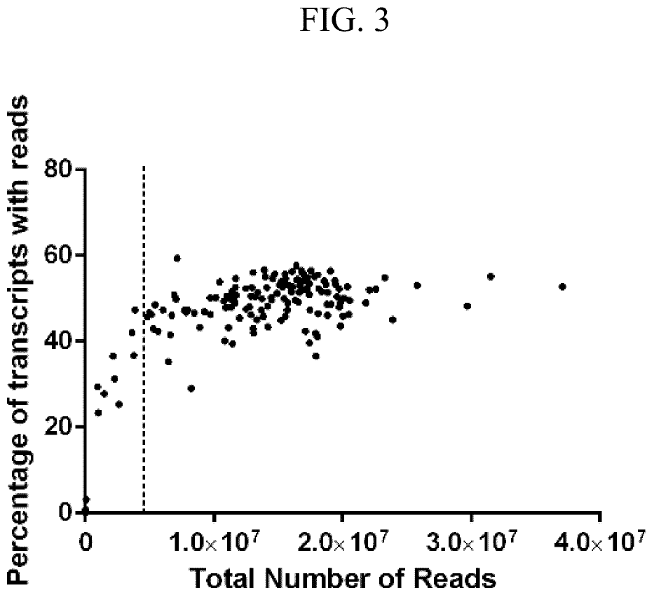

The study first removed samples with below 5 million aligned reads (see FIG. 3). The study did this to reduce the impact of samples with low expression quantification, which results in noise. This reduced the sample size to 135. From these data the study focused on African American participants (as they make up the majority of our patient population (>60%).

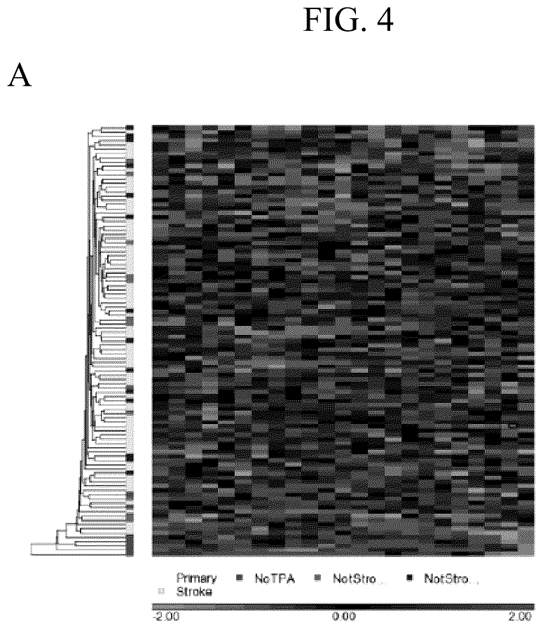

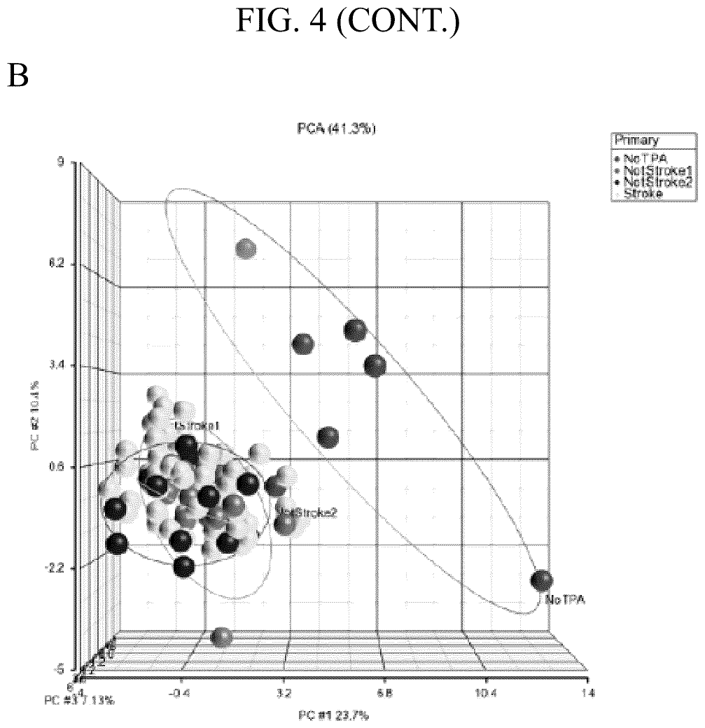

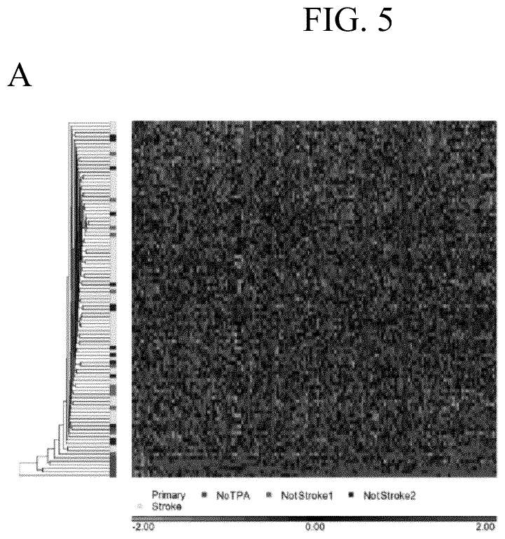

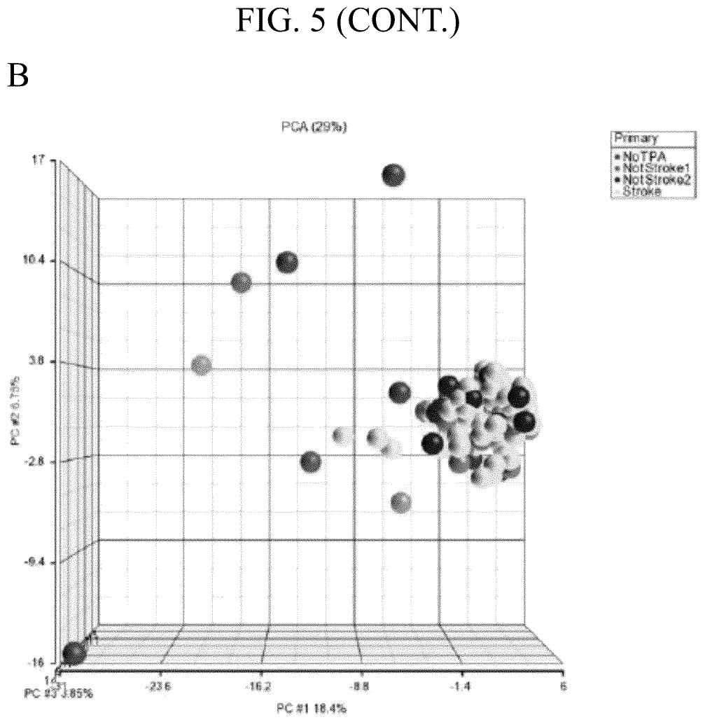

The study then performed differential expression analysis to identify hemorrhagic patients from the stroke mimic and stroke patients. The study subdivided out stroke mimics into seizures and non-seizure events. For this analysis the study had seven (7) confirmed hemorrhagic stroke patients. Regardless, the study observed significant differential expression of RNA's. Depending on the normalization scheme used the study observed 2633 genes pass an FDR 0.05, ±1.2 fold expression change when the study analyzed tpm values, and 28 when the study analyzed rpkm values. The tpm calculation creates a within data set normalization (equivalent to a z score) which appears to be effective at identifying more RNAS. The study observed similar numbers of DEGS if the study TMM normalized the rpkm values as well. The study filtered these for highly significant changes to create FIG. 4 and FIG. 5 (3-fold changes). The key issue is not to account for all statistically significant genes but rather to identify robust DEGs, which serve as a biomarker panel.

The data shows a separation of hemorrhagic patients who should not receive TPA vs other patients. There is an overlap of one patient with three ischemic stroke patients. Interestingly of these three, one patient received TPA and suffered a worsening of condition, even though CT image was adjudicated as clear. This finding is highly significant, as it shows that either CT imaging did not detect a hemorrhage or patients who have hemorrhagic conversion can be detected by this method. The three other stroke patients who did not cluster with the ischemic strokes were not treated with TPA.

The study then analyzed the same samples but for differential transcript usage. The study again observed similar patterns in the data showing differential transcript usage in the same samples with differential gene expression between hemorrhagic patients and other patient groups (Stroke and stroke mimic). This is significant for two reasons, firstly it shows both expression and isoform usage change between ischemic stroke vs hemorrhagic stroke patients. Secondly, if differential isoform usage can identify stroke patients, nanopore isoform analysis can enable a more rapid focused assessment of RNA in a rapid test.

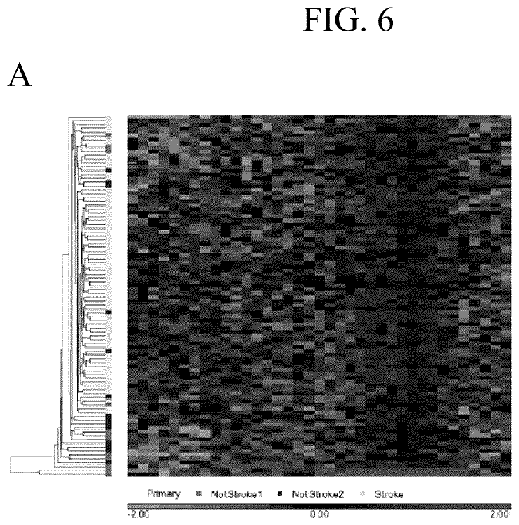

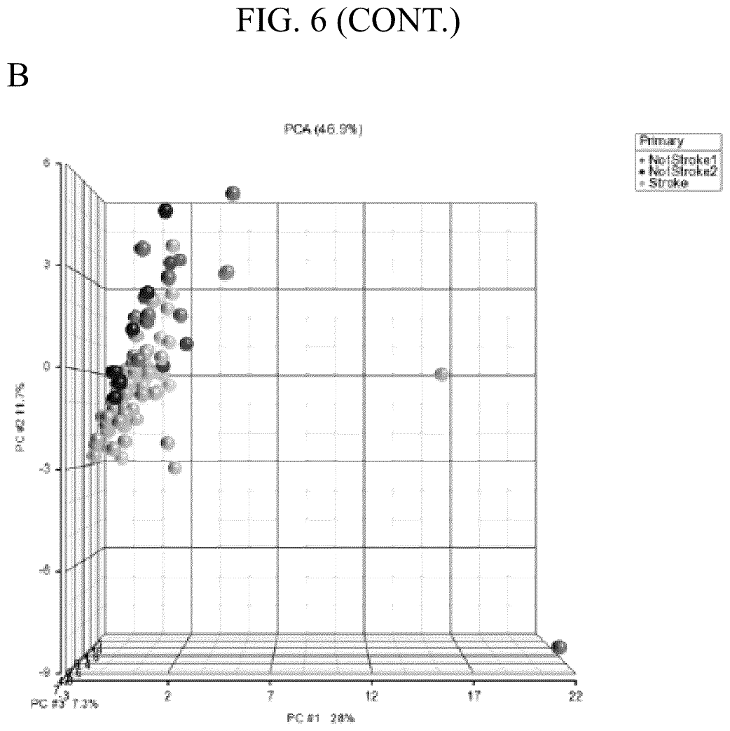

The study also investigated whether it can discriminate between stroke and stroke mimic patients, as well as identify stroke subtypes (based on TOAST criteria). The study first considered the identification of stroke vs stroke mimics. The study noticed two features of the data. First, the stroke mimics were a highly heterogenous population with clinical diagnosis of seizures, syncope, other forms of aphasia etc. As such the study split the stroke mimics into seizure and non-seizure mimics. The study then investigated Stroke vs non-stroke differences. These were not as significant as the hemorrhage vs non-hemorrhage stroke data. One potential reason is the wide variety in stroke severity. Many of the strokes have an NIHSS less than 5 (69/106 patients). Regardless using an unadjusted p of 0.001, the study observed patterns associated with stroke and stroke mimic, which could be used for a test (See FIG. 6.).

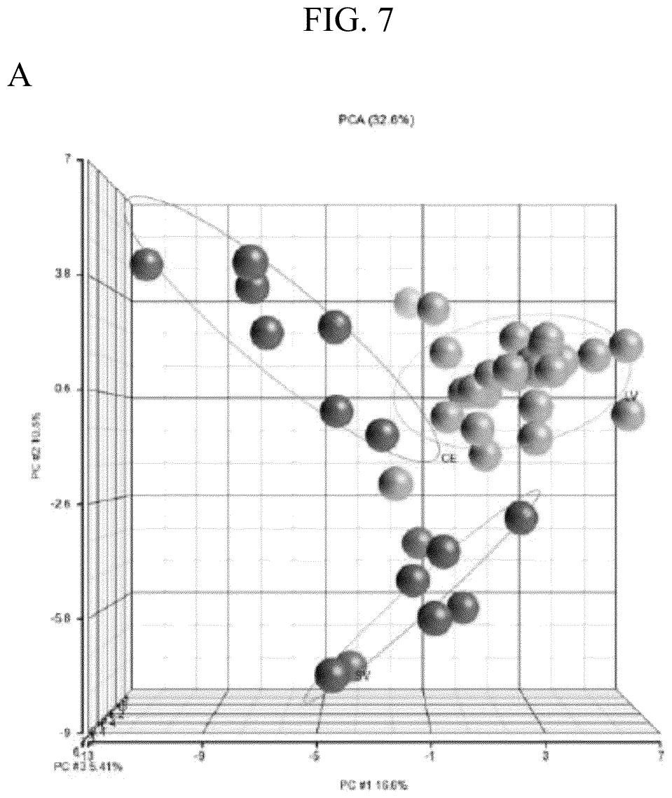

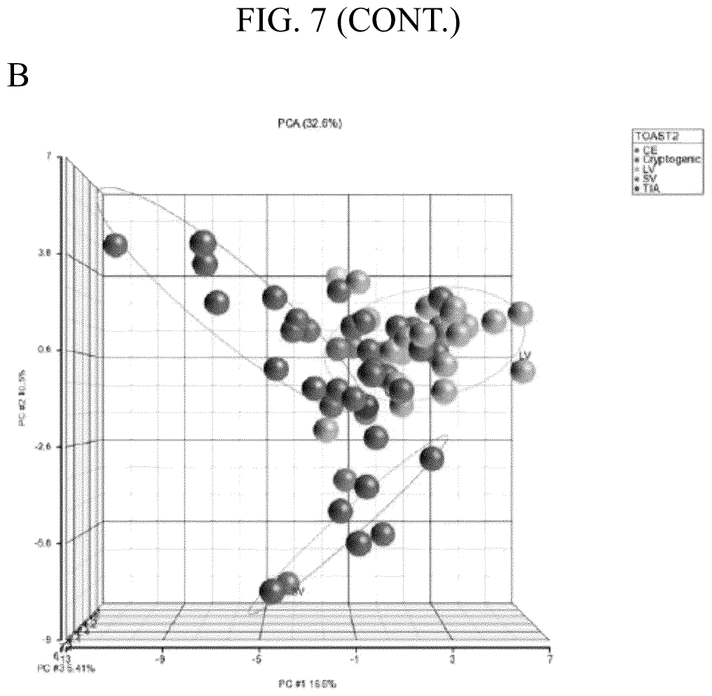

The study also performed an analysis of those patients who suffered a stroke vs TIA to determine the etiology of the TIA strokes. The study established a pattern of gene expression for Cardioembolic (CE) large vessel (LV) and small vessel (SV) strokes. FIG. 7, Panel A. The study then used these gene values and over laid the TIA data. Interestingly the TIA data aligned with either CE or LV stroke, but towards the center of the grouping. This shows that the severity of the event, or the fact that the neurological deficit is reversed within 24 h results in a smaller/weaker transcriptional response in these patients.

### Example 3: Effect of Sex and Race on Transcriptome Profile

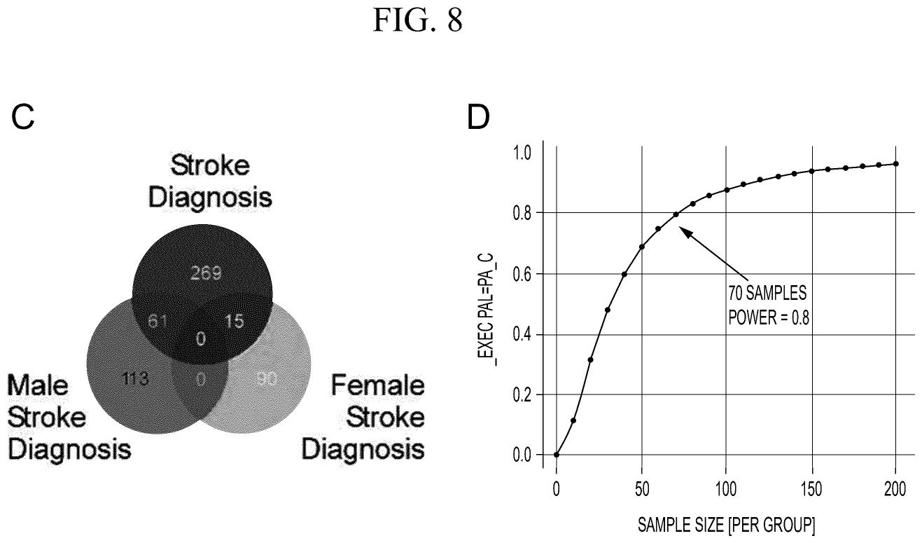

FIG. 8A-8D shows sex-specific gene expression profiles may be more accurate than a mixed sex model. Data from a previous study (Meller et al., Ann Clin Trans Neurol. 3:70-81, 2016) was refined, and only age and sex matched MCA stroke and controls used. Panel A. Hierarchical clusters of differentially expressed exon values in male only participants. Panel B. Hierarchical cluster of differentially expressed exon values in female only group. Panel C. Venn diagram showing overlap of exons in models identifying mixed sex, or sex specific changes following MCA. Note the lack of common genes in sex specific populations. Panel D. Power analysis modeling of sex specific data (female) to determine sample size (x axis) and power (y axis) to detect 2-fold changes with FDR 0.05 (modeled in SPAA).

### Example 4: Use of Gene Expression Analysis to Predict Outcome Following Stroke

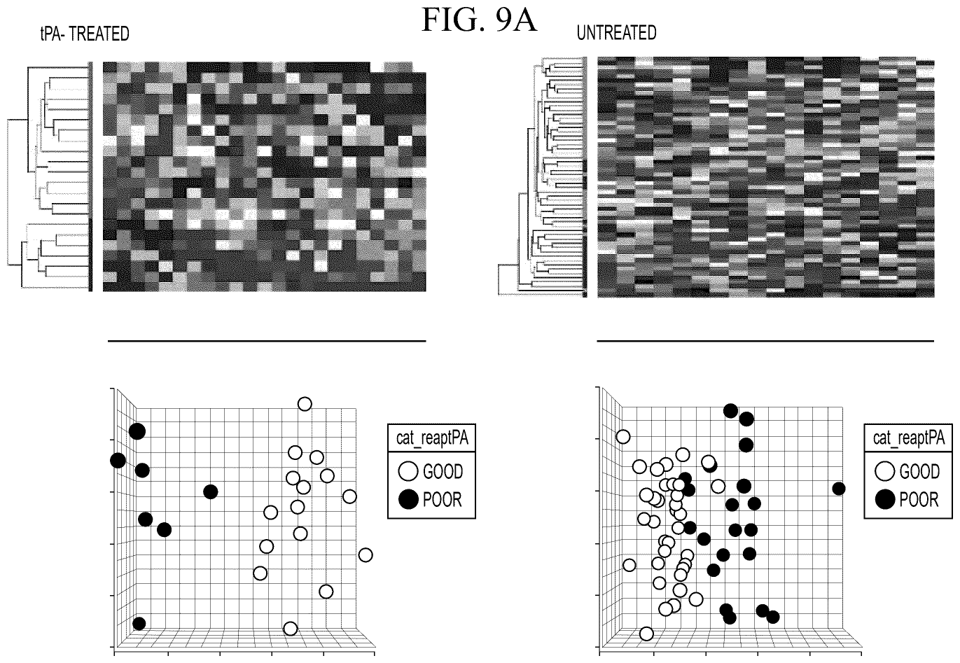

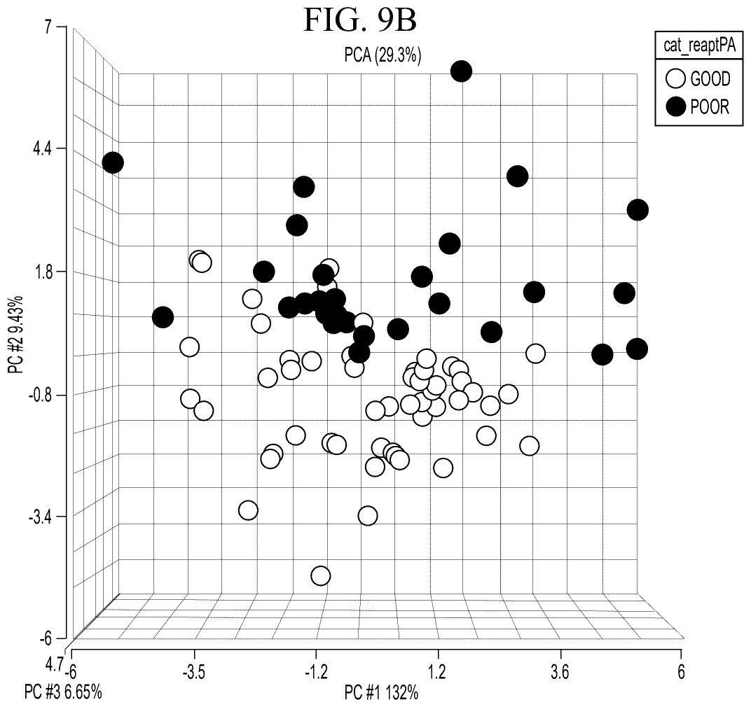

The study analyzed 120 transcriptomes from 97 Controls and 23 tPA treated patients. The study first investigated the response to tPA. While there was a reduction in NIHSS rating of untreated to treated (p<0.05 Student's t-test), there was no significant difference in the change in NIHss Rating from Admin to discharge. (FIG. 9A-9B). with an average improvement in NIHSS rating of 69%. The study used this as the cut off for a good vs poor response.

The study first analyzed all of the data, but only found weak changes in gene expression that map to a good or poor recovery from stroke. The study then separated the data by tPA treated and non tPA treated, and found well clustered gene expression changes in tPA treated samples with respect to NIH differences. However, these genes have no overlap with the genes differentially expressed between good and poor recovery from Stroke when the patient was untreated. Applying hierarchical clustering and PCA to these data the study observed a clear cluster/separation of the tPA treated patients, but not the untreated patients. Interestingly, the gene list used to separate the tPA samples shows poor clustering of the untreated data, strongly suggesting the outcome following stroke depends on both therapy and gene expression (not shown). When the study combined the list of genes used to cluster the data, and applied to all of the stroke data, the study observed strong clustering/separation of TPA-treated stroke patients, but the controls do not cluster as well. The tPA treated model was further refined by including age sex and race as factors in the linear model (3-way ANCOVA), and then selecting genes that overlap with the genes in the uncorrected data. Producing a Black patient only tPA data set resulted in a better separation of data by PCA suggesting race specific profiles may be more accurate to predict outcome.

The study used the gene list to determine whether a KNN model could identify the minimum number of genes to determine good and poor outcomes. The best model (92% accuracy) used 3 neighbors and 19 variables with a Bray-Curtis distance measure.

The final analysis approach was to try and predict the change in NIH stroke scale from admission to discharge using a lasso/ridge regression approach. Ridge regression is similar to linear regression whereas with Ridge the loss function is modified to minimize the complexity of the model. This modification is done by adding a penalty parameter that is equivalent to the square of the magnitude of the coefficients.

$$Loss function = OLS + alpha * \sum ( squared coefficient values )$$

Here OLS is the ordinary least squares (OLS) method. This method works by minimizing the sum of squares of residuals (actual value−predicted value).

This study used the control patients as the model building set and the tPA treated patients as the test set. The study performed this analysis on both all patients and African American patients. The best models created were based on single race data sets with admission NIHSS rating of >3. (the r2 for the training set was 0.91, RMSE 1.6).

The study has established the largest data set of whole blood transcriptomes from stroke patients. The present disclosure enables a blood test for ischemic stroke versus hemorrhagic stroke, which identifies patients earlier who are at very high risk of developing ischemic stroke. These patients can be identified when their symptoms are less severe, so any therapy may be able to at least halt the progression of the disease. The blood test is effective in these patients and can be a routine screening tool for emergency admissions to hospital.

### Example 5: Modeling Process

Seq data was aligned to the human reference genome using STAR and Bowtie2 (part of the Ion Torrent software RNA-Seq protocol). All subsequent analysis was performed using the Partek Genomics Suite v 7.0 running on a dedicated Dell Precision T7600 workstation (80 Gb RAM: 40 Tb storage). RNA-Seq data files (BAM files) were used to generate gene expression values (reads), exon expression and transcript expression values. Copies of Bam files were transferred to a local encrypted database for storage (QNAP TVS1828T-60 Tb storage (RAID6)). The study maintained a de-identified database of clinical phenotype data alongside each transcriptome.

The study used differential expression analysis to identify classifiers (gene, transcript or exons) for use in modeling to predict a clinical data element (stroke phenotype etc.). The clinical data elements were determined by a team of neurologists by reviewing the patient's charts. The RNA-Seq data set was split into a MODELING and a VALIDATION data set. Differential exon expression for a given clinical phenotype (i.e. stroke vs. no stroke) was determined in the MODELING data set using Partek Genomics suite (ANOVA, ±2.0-fold change FDR p<0.05). This list of exons was then used by the prediction module of Partek to identify models using smaller subsets of expression values with the highest accuracy, sensitivity and specificity using support vector machine modeling. Models are then refined using cross-validation analysis (two-level and bootstrap). Models with the highest normalized correct rate are forwarded to validation testing. The list of exon classifiers identified in the modeling dataset was extracted from the validation data set, and then tested to determine how accurately they predicted the diagnosis of the validation data. These data were subject to receiver operator curve (ROC) analysis. The primary outcomes were a measure of accuracy (%) sensitivity, sensitivity, and odds ratio . . . .

The contents of all references, patents, and published patent applications cited throughout this application, as well as the Figures and Tables, are incorporated herein by reference.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present application, and it is not intended to detail all those obvious modifications and variations of it which will become apparent to the skilled worker upon reading the description. It is intended, however, that all such obvious modifications and variations be included within the scope of the present application, which is defined by the following claims.

What is claimed is:

1. A method for assessing stroke suspects, comprising the steps of:
extracting RNA from a blood sample of a stroke suspect;
generating a transcriptome profile of the stroke suspect based on the RNA extracted from the blood sample; and
analyzing the transcriptome profile of the stroke suspect to generate a report indicating whether the stroke suspect suffered a stroke and, in the case that the stroke suspect suffered a stroke, whether the stroke is an ischemic stroke or a hemorrhagic stroke.

2. The method of claim 1, wherein the blood sample is collected at admission of a medical facility.

3. The method of claim 1, wherein the analyzing step comprises comparing the transcriptome profile of the stroke suspect to established transcriptome profiles of stroke patients and determining the stroke subtype.

4. The method of claim 1, wherein the report indicates that the stroke suspect suffered a stroke and wherein the outcome includes a recommendation for appropriate treatment or prevention regime for the stroke.

5. The method of claim 4, wherein the report indicates that the stroke suspect experienced an ischemic stroke and wherein the recommended treatment is tissue plasminogen activator (tPA) treatment or similar pharmacological clot removing therapy.

6. The method of claim 1, further comprising the step of:
in the case that the stroke suspect suffered a stroke, treating the stroke suspect with a stroke treatment.

7. The method of claim 6, wherein the stroke suspect suffered an ischemic stroke and wherein the stroke suspect is treated with tPA or other pharmacological agent.

8. The method of claim 1, wherein the RNA extracted from the blood sample is not enriched for mRNA.

9. A method for treating stroke patients with tissue plasminogen activator (tPA), comprising the steps of:
extracting RNA from a blood sample of a stroke suspect;
generating a transcriptome profile of the stroke suspect based on the RNA extracted from the blood sample; and
comparing the transcriptome profile of the stroke suspect to an established transcriptome profile of ischemic stroke patients; and
determining whether the stroke suspect suffered an ischemic stroke based on a result of the comparing step; and,
treating the stroke suspect with tPA, if the stroke suspect suffered an ischemic stroke.

10. The method of claim 9, wherein the stroke suspect is determined to have suffered an ischemic stroke, when the transcriptome profile of the stroke suspect matches the established transcriptome profile of ischemic stroke patients.

11. The method of claim 9, further comprising the step of comparing the transcriptome profile of the stroke suspect to an established transcriptome profile of hemorrhagic stroke patients.

12. The method of claim 11, wherein the stroke suspect is determined to have suffered an ischemic stroke, when (1) the transcriptome profile of the stroke suspect matches the established transcriptome profile of ischemic stroke patients and (2) the transcriptome profile of the stroke suspect does not match the established transcriptome profile of hemorrhagic stroke patients.

13. A method to predict response to therapy in stroke suspects, comprising the steps of:
extracting RNA from a blood sample of a stroke suspect;
generating a transcriptome profile of the stroke suspect based on the RNA extracted from the blood sample; and
analyzing the transcriptome profile of the stroke suspect to generate a report indicating whether the stroke suspect suffered a stroke and, in the case that the stroke suspect suffered a stroke, predicting the stroke suspect's response to a stroke therapy.

14. The method of claim 13, wherein the stroke therapy is tPA treatment or mechanical removal of a blood clot.
